(19) United States
(12) Patent Application Publication
(10) Pub. No.: US 2015/0027441 A1
(43) Pub. Date: Jan. 29, 2015

(54) NEBULIZER DEVICE FOR MEDICAL AEROSOLS

(71) Applicant: La Diffusion Technique Francaise, Saint Etienne (FR)

(72) Inventors: Laurent Vecellio-None, Chambray Les Tours (FR); Gilles Chantrel, Saint Etienne (FR); Michel Massardier, Saint Etienne (FR)

(21) Appl. No.: 14/373,624

(22) PCT Filed: Jan. 18, 2013
(86) PCT No.: PCT/FR2013/050114

§ 371 Date: Jul. 21, 2014

(30) Foreign Application Priority Data
Jan. 20, 2012 (FR) 1250569

(51) Int. Cl. A61M 16/20; A61M 15/00; A61M 16/14

(52) U.S. Cl. CPC A61M 16/208; A61M 16/14; A61M 15/0085; A61M 15/0026; A61M 15/0088; A61M 2205/0266
USPC 128/200.16; 128/200.14

(57) ABSTRACT

A nebulizer device including a mesh aerosol generator combined with an inhalation chamber formed with three openings and a single one-way expiratory valve, a first opening being connected to the patient and used for the transport and administration of the particles from the device to the patient, the said first opening being placed upstream of the aerosol generator, a second opening placed upstream of the device aerosol generator allowing the passage of air from the outside of the chamber to the inside of the inhalation chamber, and allowing the complete ventilation of the inside of the inhalation chamber, a third opening, placed downstream of the aerosol generator and upstream of the said first opening, provided with a single one-way expiratory valve allowing the exhalation of the air from the patient through this third opening, the said third opening being closed by means of the one-way expiratory valve during the inhalation phases and open during the exhalation phases, and whereby the resistance of the said third opening, combined with that of the expiratory valve is less than the resistance of the second opening.

### BACKGROUND

1. Technical Field

The invention concerns the technical sector of medical aerosol generating systems.

2. Description of the Related Art

Medical aerosol generating systems are designed to transform liquid or powder medicine into an aerosol for administration to the airways.

There are various medical aerosol generating systems on the market in derived forms of devices with pneumatic or ultrasonic controls, with meshes in particular, as well as pressurized canisters with a dosimetric valve. Nebulizer devices are used for projecting large quantities of varied medicines directly into the airways of patients. They have a fast and selective therapeutic action on the pathological site itself. Nebulization can be described as the transformation of liquid medicine into an aerosol medicine. The term “nebulize” is used as a synonym for “transforming a liquid into an aerosol”. The nebulizer device has a container in which the liquid to be nebulized is added, a nebulization chamber where the aerosol is produced, and a source of energy—pneumatic or piezo-electric—which creates the aerosol.

Today, three nebulization principles are used for producing an aerosol medicine: pneumatic nebulization, ultrasonic nebulization and, more recently, mesh nebulization.

Mesh nebulizer devices are new generation nebulizer devices. These systems are silent and can be portable and small. They consist of an electronic power supply unit connected to the nebulizer device. The electronic power supply unit can run on the mains or on batteries. The aerosol generator comprises a mesh pierced with a large number of microscopic holes (between 1000 and 6000 holes depending on the model). These holes, measuring between 2 μm and 10 μm in diameter, are obtained by electrophoresis or by laser drilling into the mesh. The passage of the liquid medicine through the mesh transforms the liquid into aerosol droplets of an equivalent size to the size of the holes in the mesh.

At present, there are two main types of mesh aerosol generators, the fixed mesh and the vibrating mesh.

The fixed mesh aerosol generator uses a piezo-electric device in contact with the liquid medicine. The vibration of the piezo-electric device (between 50 kHz and 500 kHz) is transmitted to the liquid, forcing it to pass through the holes in the mesh and expelling the breathable mist of droplets from the mesh. The aerosol produced is then mobilized by the patient away from the nebulizer device.

The vibrating mesh aerosol generating uses a piezo-electric device around the mesh to make it vibrate (between 50 kHz and 500 kHz). The medicine solution which is thus in contact with the mesh is drawn in through the conical holes when it deforms towards the solution container. When the mesh deforms towards the outside, the solution then on the edge of the mesh breaks free to form breathable particles. The aerosol is then mobilized by the patient outside the nebulizer device. Using a system like this, only a negligible amount of medicine is lost in the container and the flow rate of the aerosol produced is comparable to that of ultrasonic nebulizer devices (approximately 0.5 ml/min).

The medicine aerosol generating systems consist of an aerosol generator as such and an interface between the aerosol generator and the patient. The aerosol generator is the generation source of the aerosol. The patient interface transports the aerosol mist from the generator towards the patient (e.g., mask, mouthpiece, nosepiece, mechanical ventilator circuit, intubation probe, tracheal probe, etc.). Some aerosol medicine generating devices producing an aerosol continuously can generate a loss of medicine into the atmosphere during the expiratory phase of the patient. Indeed, when the patient exhales into the generating device, or no longer inhales from the generating device, the aerosol can escape freely from the generating device. This is typical, for instance, of the Aeroneb or Micro Air nebulizer devices. The aerosol is produced by the aerosol generator in a volume with two openings: the first opening is designed to be connected to the patient and the second opening is free, allowing the passage of the air inhaled and exhaled by the patient. Accordingly, during the inhalation phase of the patient, the air inhaled by the patient passes through the second opening and carries the aerosol towards the patient. When the patient exhales through the mouth, the air exhaled carries with it the aerosol produced by the aerosol generator which is expelled into the ambient air through the second opening. Accordingly, the nebulizer device generates considerable aerosol losses during the exhalation phase of the patient.

To minimize the losses of aerosol during the exhalation phase of the patient, an intermediate zone can be added between the aerosol generating zone and the interface. This intermediate zone is called the storage zone. The storage zone and the interface together then form the inhalation chamber designed to store the aerosol during the exhalation phases of the patient and to administer the stored aerosol during the patient respiratory phases. Various technical means ensure the operation of the inhalation chamber according to the breathing phases of the patient.

The patent filed by Pari (WO/0134232A1) describes a mesh nebulizer device with an inhalation chamber having a double valve system so that during the exhalation phase, the air exhaled by the patient is expelled through a first expiratory valve without passing through the storage zone and so that during the inhalation phase, the air entering the storage zone through the inhalation valve transports the aerosol towards the patient. The inhalation valve is closed during the exhalation phase and open during the inhalation phase; the expiratory valve is open during the exhalation phase and closed during the inhalation phase.

The patent filed by the Novartis laboratory (WO2010008424) describes a chamber using a single valve in the storage zone and a resistance to the flow of air, hereinafter called the air resistor, at the interface (e.g., filter). Under these conditions, the air exhaled by the patient is expelled by the filter without going through the storage zone and the air inhaled passes essentially through the storage zone to transport the aerosol to the patient via the interface. This way of operating the inhalation chamber can only be ensured by using a low resistance valve with the consequence of having a device with high resistance to exhalation and low resistance to inhalation.

The patent filed under the name of la Diffusion Technique Française (EP 1 743 671) describes an inhalation chamber not using valves, but such that the exhaled and inhaled air circuits are different. Under these conditions, the air exhaled by the patient is expelled from the chamber without going through the storage zone and the air inhaled goes through the storage zone to transport the aerosol to the patient via the interface. In its operation, this chamber embodies a constraint of there being no resistance provided by the openings in order not to create any overpressure in the chamber with the consequence of the device having low resistance to exhalation and to inhalation.

Furthermore, it is known that the inhalation rate of the patient is an important parameter. A low inhalation rate improves pulmonary deposition compared to a high inhalation rate. In fact, the particles transported at a lower inhalation rate will move at a lower speed, thus limiting the physical phenomenon on the impact in the oropharyngeal system. Traveling at a low speed, these particles will be able to enter the lungs without being deposited in the upper airways. For instance, the use of an air resistor during the inhalation phase of the patient limits the flow of air inhaled to improve the treatment. Physically, this resistance is obtained by using a small diameter opening which resists the flow of the air drawn in by the patient. This opening is placed in the storage zone upstream of the aerosol generator to limit the speed of the particles as they penetrate into the patient. By comparison, a small diameter opening downstream of the aerosol generator would result in the increased speed of the particles administered to the patient. According to the previous descriptions of the operating principles of a mesh nebulizer device using inhalation chambers, only the use of a costly double valve system (WO/0134232A1) allows the limiting of the patient inhalation rate by means of a resistor at the inhalation valve, upstream of the aerosol generator. The operating principles of the inhalation chambers using a valve, or no valve, currently will not limit the inhalation rates by the use of an air resistor placed upstream of the aerosol generator.

### BRIEF SUMMARY

The Applicant is one of the leaders in the manufacture and sale of this type of device.

Confronted by this situation, the Applicant turned towards a different design of this type of device, reducing the losses of the aerosol by storing it during the patient exhalation phase and ensuring the limiting of the patient's inhalation rates and reducing the costs of industrialization.

The solution discovered runs against the grain of known solutions being in complete opposition to them with respect to the implementation and operation of the corresponding nebulizer devices.

According to a first characteristic, the nebulizer device includes a mesh aerosol generator associated with an inhalation chamber, and is noteworthy in that the inhalation chamber has three openings and a single one-way expiratory valve, with a first opening connected to the patient for transporting particles from the device to the patient, and the said first opening being placed downstream of the aerosol generator, the second opening being placed upstream of the aerosol generation of the device allowing air to pass from outside the chamber to the inside of the inhalation chamber, the third opening, placed downstream of the aerosol generator and upstream of the first opening, is provided with a single one-way expiratory valve allowing air to be exhaled by the patient through this third opening, the said third opening being closed by means of the one-way expiratory valve during the inhalation phases and opened during the exhalation phases and whereby the resistance of the said third opening, combined with that of the expiratory valve, is less than the resistance of the second opening, in order to limit the escape of the particles produced in the inhalation chamber through the second opening.

According to the invention, the inhalation chamber is designed so that the average resistance of the second opening is higher than the average resistance of the said third opening and of the expiratory valve for the various exhalation rates, thus allowing the separation between the inhaled and exhaled air circuits in the device.

The chosen solution is therefore opposite to that of the operation of the inhalation chambers referred to previously.

Accordingly, using the solution of the invention, the exhalation rate of the patient can be modeled by a sinusoidal function depending on time and in which the first milliliters of air exhaled by the patient correspond to flow values of a few liters/minute. These low flow values are in keeping with the sensitivity of the low resistance mechanical valves available in the market (resistance less than 0.07 cm H20 min/L). Accordingly, using a low resistance valve will ensure the operation of the system as soon as the patient begins to exhale, and maintain its operation during the continuation of the patient exhalation time. From these considerations, the minimum exhalation rate for the resistance of the third opening and the resistance of the expiratory valves to be less than the resistance of the second opening can be set starting from 3 L/min to ensure the opening of the valve as soon as the patient begins to exhale, and maintain the system in operation for more than 90% sign of the patient exhalation time. The maximum expiration rate of a standard patient breathing calmly corresponds to 30 L/min (for an exhalation time of 2 seconds and a current value of 500 mL) but can be up to 600 L/min during forced exhalation. Accordingly, for a calm inhalation, the resistance of the second opening is advantageously at least 10 times greater than the resistance of the valve and the resistance of the third opening for an exhalation rate of 30 L/min. The second opening can comprise one orifice or several orifices.

Advantageously, the aerosol generator is placed in the upper part of the chamber to limit the loss of the aerosol in the chamber due to sedimentation phenomena. The introduction of the aerosol by an aerosol generator in the side part of an inhalation chamber increases the losses of aerosol in the device (WO/0134232A1). The particles brought in from the side to the central or bottom part of the storage zone will sediment faster than the particles brought into the upper part of the storage zone. The first opening, designed to be connected to the patient, is also placed advantageously in the top part of the chamber, preferably near the aerosol generator, to ensure that the total emptying of the chamber of the each inhalation. If the system does not empty entirely, it will increase the successive concentration of the aerosol in the chamber and could increase deposition on the surface of the storage area by diffusion. The third opening comprising the valve is preferentially placed near the first opening to limit the dead space of the system which would cause the asphyxia of the patient by re-breathing, and by not evacuating it the totality of the aerosol from the system. The maximum volume included between these two openings can be defined as the standard anatomic dead volume of 200 mL for an adult. Therefore, the third opening comprising the valve is advantageously placed at the patient interface comprising for instance, a mouthpiece, a face mask or a nose piece, but can also be separated from the patient interface.

The second opening, designed to allow the air to penetrate into the inhalation chamber during the inhalation phase is placed so that it allows the complete ventilation of the storage area to transport all the aerosol stored in the storage area towards the patient.

The shape of the storage zone plays a part in the deposition by the impact of the particles against the inner walls during their transport. A cylindrical storage zone profiled on a vertical axis, for instance, decreases this phenomenon. The shape of the storage zone according to the invention is particularly well-suited to the diffusion of the aerosol and limits deposition by impaction. The use of a cylindrical shape ensures good transport of the aerosol out of the storage zone and limits the air re-circulation zones, that is, the areas with poor ventilation in the storage zone. What is more, to ensure the advantageous use of the cylindrical shape, combined with the second opening, a second cylinder enclosing the first cylinder can be used. The diameter on this second cylinder is larger than the first cylinder and it has only the second opening at the apex to ensure the total ventilation of the inside of the inhalation chamber and limits the leakage of particles out of the system through this second opening, when the patient pauses in its breathing. The column of air in the space included between the first cylinder and the second cylinder operates at a certain overpressure, limiting the movement of the particles from the first cylinder into the second cylinder.

In this way, the device according to the invention is particularly advantageous because the combination of these characteristics limits the loss of aerosol in the ambient air by storing it during the exhalation phase and limiting losses by deposition on the walls. Combined with the relative positions of the air inlet and outlet openings of the aerosol, this storage zone therefore increases the quantity of aerosols is stored between each inhalation and limits its deposition on the walls. It also allows the generated aerosol to be received at a relatively high speed without any losses on the walls. The volume in which the aerosol is projected is included between 40 mL and 500 mL. It is large enough for the particles generated at a high speed to have the time to be slowed down by the action of air friction, thus limiting their deposition by impact on the walls of the complementary means. This storage zone also allows the aerosol to be concentrated during the patient exhalation phase, thus increasing the quantity of active principle inhaled by the patient on each inhalation, and thus increasing the rate of the system. In addition, the use of openings with different resistance values ensures high resistance to inhalation and a low resistance to exhalation of the patient with the consequence of reducing the inhalation rate, decreasing the oropharyngeal deposition and increasing in the deposition in the lungs.

Therefore, not only does the invention increase the efficiency of the aerosol generation system but it also improves the efficiency of the deposition of the aerosol in the patient's lungs by resisting the flow of air during the inhalation of the patient.

### BRIEF DESCRIPTION OF THE SEVERAL VIEWS OF THE DRAWINGS

These and other characteristics are described in the continuation of this document.

FIG. 1 is a schematic illustration of the basic principle of the invention of the device shown as a profile view,

FIG. 2 is a schematic representation of the device shown in profile, in operation, including a mouthpiece type interface, during the exhalation phase with the device comprising two cylinders defining the particle storage zone,

FIG. 3 is a schematic representation of the device shown in profile, in operation, including a mouthpiece type interface, during the inhalation phase with the device comprising two cylinders defining the particle storage zone.

FIG. 4 is a schematic representation of the device shown in three dimensions, including an orthogonal mouthpiece type interface, during the exhalation phase with the device comprising two cylinders defining the particle storage zone,

FIG. 5 is a schematic representation of the device shown in three dimensions, including an orthogonal mouthpiece type interface, during the inhalation phase, with the device comprising two cylinders defining the particle storage zone,

### DETAILED DESCRIPTION

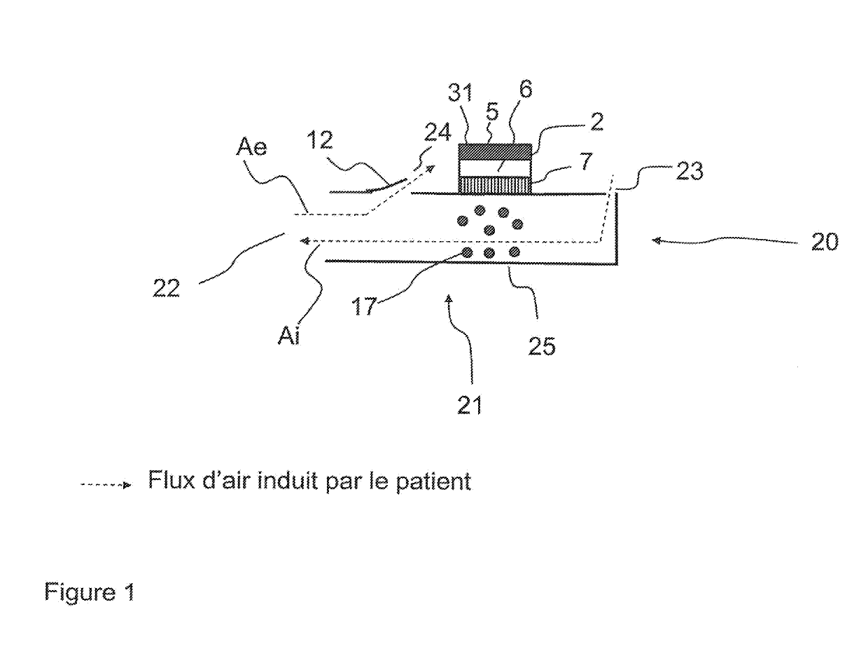

FIG. 1 is an illustration of the basic principle of the invention, shown in profile. The device (20) includes an aerosol generator (2) ensuring the generation of particles (17) in an inhalation chamber (21) comprising three openings, a first opening (22) designed for connection to the patient, a second opening (23) allowing outside air to pass through the inhalation chamber (21) and a third opening (24) with a one-way expiratory valve (12). The aerosol generator (2) is a fixed mesh generator with a container (5) to which the medicine (6) is added and a mesh (7) associated with a piezo-electric device whose vibration frequency is included between 50 kHz and 500 kHz and a mesh (7) kept separate from the piezo-electric device and used for transforming of the liquid medicine (6) into medicine particles (17) in the storage zone (25) of the inhalation chamber (21).

Advantageously, the expiratory valve (12) is made of a shape-memory material closing it at ambient pressure and opening it at a pressure which is greater than 3 cm H20 in association with device (20). For instance, the expiratory valve (12) has resistance of less than 0.03 cm H20 min/L for an exhalation rate of 30 L/min and is made of silicon or elastomer. The valve can also be made of a solid non-deforming material and be connected to the patient interface via a rotation axis hinge. This hinge closes by gravity and opens under slight overpressure. In this case, the resistance of the expiratory valve (12) is even less and offers resistance of less than 0.001 cm H20 min/L for an exhalation rate of 30 L/min. The third opening (24) also offers low resistance. In the light of the standard exhalation rate, and counted during an inhalation session, the third opening (24) has a minimum preferential diameter of 5 mm. The second opening (23), allowing outside air to pass into the chamber (21) has a resistance which is greater than the resistance of the third opening (24) associated with the valve (12).

Advantageously, the resistance of the second opening (23) is 10 times greater than the resistance of the third opening (24) associated with the valve (12) for an exhalation rate of 30 L/min. This second opening (23) can comprise one orifice, or several orifices. Preferentially, the diameter of the orifice is less than 20 mm. Furthermore, to limit the risks of these orifices being obstructed, because of their small sizes, and to ensure their thorough cleaning after each inhalation session, the diameter of an orifice is at the least 1 mm. For instance, the equivalent resistance of six orifices having a diameter of 2 mm is 6 cm H20 min/L for a rate of 30 L/min. In this way, during the exhalation phase of the patient, the resistance of the valve (12) and of the third opening (24) is less than the resistance of the second opening (23) so that the air exhaled (Ae) by the patient through the first opening (22) is expelled preferably by the third opening (24) without passing through the storage zone (25). The particles (17) produced by the aerosol generator (2) are stored in the storage zone (25) during the exhalation phase of the patient. During the inhalation phase of the patient, the expiratory valve (12) closes the third opening (24) and the air drawn in (Ai) by the patient passes through the second opening (23) carrying with it the particles (17) produced and stored in the storage zone (25) towards the patient through the first opening (22).

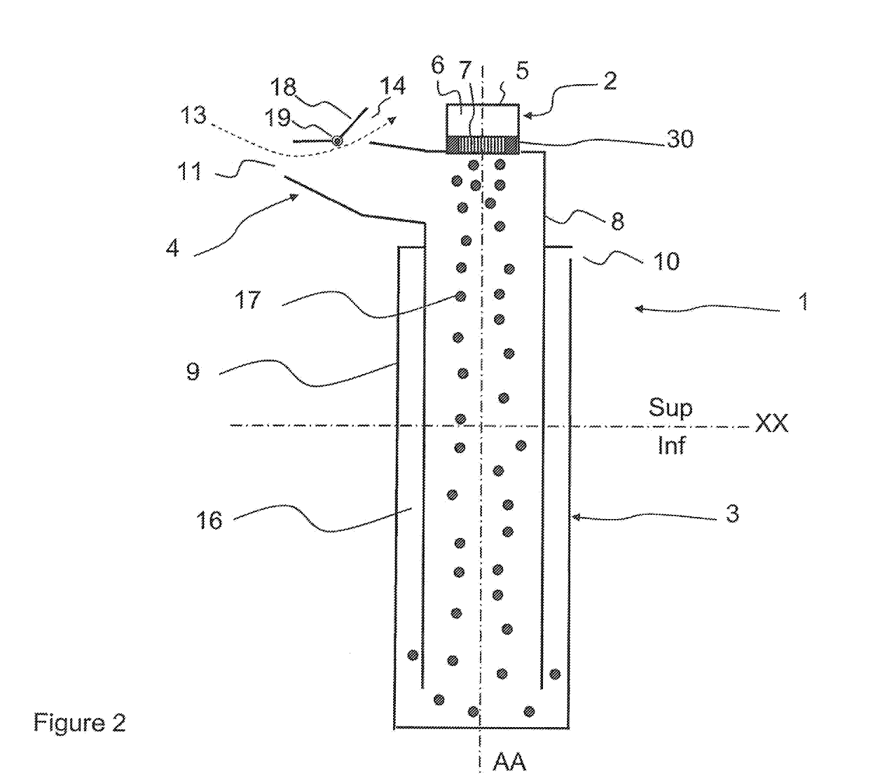

FIG. 2 illustrates the device (1) according to an optimized embodiment of the invention. The device includes an aerosol generator (2), a storage zone (3) and a mouthpiece type interface (4). The aerosol generator is a vibrating mesh generator known in a previous embodiment, including a container (5) to which the medicine (6) is added and a mesh (7) made to vibrate by a piezo-electric device (30) at a frequency included between 50 kHz and 500 kHz, transforming the liquid medicine into medicine particles (17).

Advantageously, the aerosol generator is placed in the top upper part (Sup) of the storage area (3) to limit the loss of aerosol into the storage area because of sedimentation. This upper part (Sup) is defined with respect to a middle transverse axis (XX) through the height of the device. Preferentially, the storage zone (3) consists of two cylinders each included in one another and profiled and arranged on a vertical axis (AA). The first cylinder (8) is open at either end. The second cylinder (9) containing the first cylinder (8) has only one opening (10) in the top section. The shape of the cylinder according to the invention is particularly well-suited to the diffusion of the aerosol and limits deposits by impaction. Because of the flow of the aerosol generator and the inhalation rate of the patient, preferentially, the first cylinder (8) has a diameter included between 20 mm and 100 mm. Advantageously, the diameter of the second cylinder (9) is three times smaller than the diameter of the first cylinder (8) so that the distance between the walls of each of the cylinders (8 and 9) is smaller than the diameter of the first cylinder (8) to limit the volume of aerosol stored between the two walls (16). The aerosol which is stored between the two walls (16) represents a high rest of impact in the lower part of the second cylinder (9) during its transport in the inhalation phase. In addition, to limit the sedimentation of the aerosol at the bottom of the device (1), the storage zone is at least 40 mm high and the space between the bottom of the first cylinder (8) and the bottom surface of the second cylinder (9) is at the least 1 mm.

The mouthpiece type interface (4) is placed on the upper side part of the storage zone (3), preferentially near the aerosol generator to ensure the total emptying of the chamber after each inhalation.

The interface (4) has an opening (14) and a non-deformable expiratory valve (18) provided with a rotation axis hinge (19) which opens the opening (14) during the exhalation phase and closes the opening (14) during the inhalation phase and the breathing pause phases.

FIG. 2 illustrates operation in this optimized configuration of the invention during the patient exhalation phase. The liquid medicine (6) is added to container (5) of the aerosol generator and the particles (17) are produced through the vibrating mesh (7) along axis (AA) of the cylinder (8). Because of the weight and the initial velocities of the particles, downward force towards the bottom of the device is applied to the particles (17). Conversely, pressure force from the head of air included between the two cylinders (8 and 9) is applied towards the bottom of the space (16). This force, in opposition to the first force, limits the movement of the particles towards the space between the two cylinders (16) and also towards the opening (10) in contact with the outside air. The patient breathes out through the mouth into the device through the opening (11) at the end of the interface (4). The air exhaled (13) by the patient creates a slight overpressure in the device. The resistance of the opening (14) and of the valve (18) is lower than the resistance of the opening (10) so that the valve (18) lifts and opens the opening (14). The air exhaled (13) by the patient is expelled from the device through the opening (14). The exhaled air (13) does not pass through the storage zone (3) and the particles produced (17) are stored in the storage zone (3) for the following inhalation.

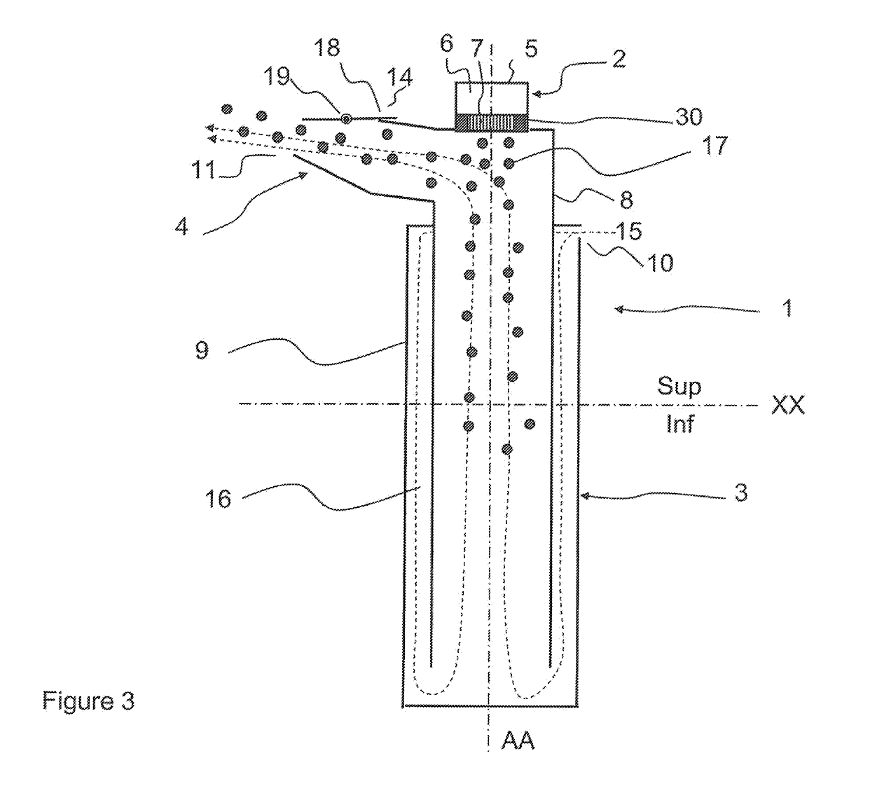

FIG. 3 illustrates the operation of the device in an optimized implementation of the invention during the inhalation phase of the patient. The device (1) shown in FIG. 3 is identical to the device (1) shown in FIG. 2. The particles (17) are generated in the storage zone (3) by the aerosol generator (2). The patient is connected through the opening (11) of the interface (4) so that, during the inhalation phase, the valve (18) closes the opening (14). The air inhaled (15) by the patient connected to the opening (11) of the interface (4) enters the device (1) through the opening (10) while creating a resistance to inhalation so as to limit the inhalation race of the patient, keeping it to a low value. The air (15) enters the second cylinder (9) then the first cylinder (8) from which it is inhaled by the patient through the opening (11). In passing through the device (1), the air (15) carries with it the particles (17) generated by the aerosol generators (2) and stored in the storage zone (3) during the exhalation phase (FIG. 2) prior to this inhalation phase (FIG. 3).

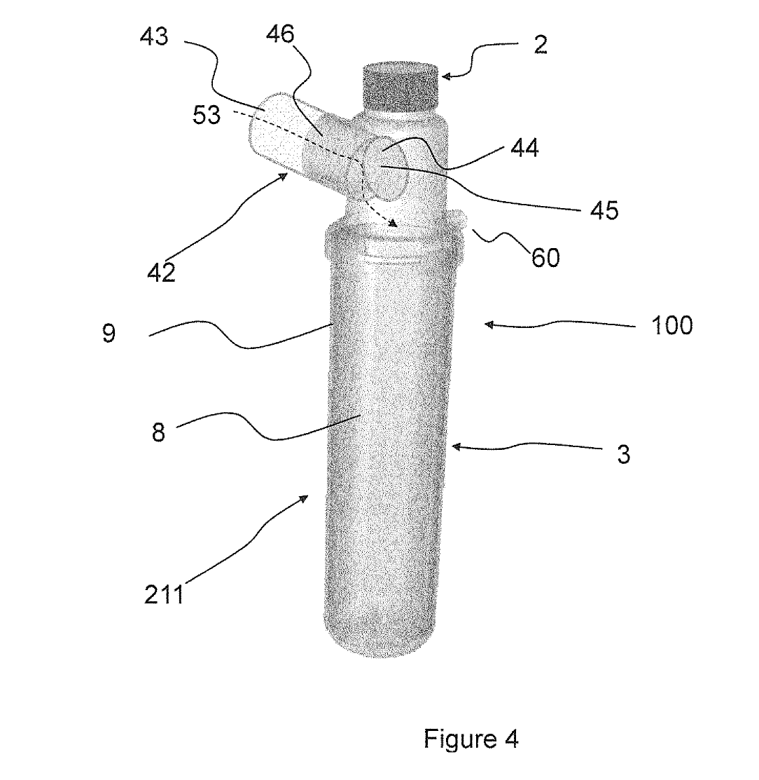

FIG. 4 illustrates the device (100) according to an embodiment of the invention with an orthogonal type mouthpiece (42). The device (100) includes an aerosol generator (2) which generates particles (17) in an inhalation chamber (211). This inhalation chamber (211) consists of a storage zone (3) and a mouthpiece type interface (42). The aerosol generator is a vibrating mesh generator (2).

The orthogonal mouthpiece type interface (42) has an opening (43) designed for connection to the patient, an opening (44) with a low resistance one-way expiratory valve (45) allowing the patient to breathe out through the valve (45), and a passage section (46) allowing the aerosol to pass from the storage zone (3) to the mouthpiece (42). The axis connecting the passage sections of the openings (43) and (44) is perpendicular to the axis of passage section (46). The air exhaled (53) by the patient is directed in a straight line towards opening (44) and creates slight overpressure inside the device. The resistance of the opening (44) and of the valve (45) is lower than the resistance of the second opening (60) so that valve (45) lifts and opens the opening (44). The air exhaled (53) by the patient is directed straight towards opening (44) and expelled from the device (1) through opening (44). The exhaled air (53) does not pass through the storage zone (3) and the particles produced are stored in the storage zone (3) for the following inhalation.

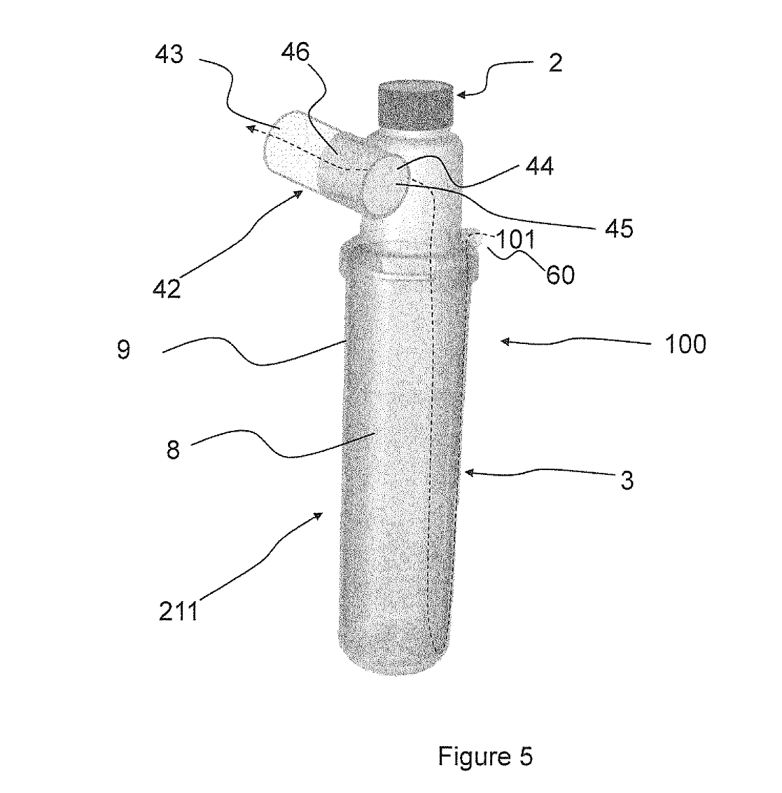

FIG. 5 illustrates the operation of the device in an embodiment of the invention comprising an orthogonal mouthpiece interface, during the patient's inhalation phase. The device (100) shown in FIG. 5 is identical to the device (100) shown in FIG. 4. The particles are generated in the storage zone (3) by the vibrating mesh aerosol generator (2). The patient is connected to the opening (43) in interface (42). During the inhalation phase, the valve (45) closes the opening (44). The air inhaled (101) by the patient, connected to opening (43) of interface (42) enters the device (100) through the second opening (60) while establishing resistance to inhalation and thus limit the inhalation flow of the patient, keeping it to a lower value. The air (101) enters the second cylinder (9) and then the first cylinder (8) and is inhaled by the patient through the passage section (46) then opening (43). In passing through the device (100), the air (101) carries with it the particles generated by the aerosol generator (2), stored in the storage zone (3) during the exhalation phase (FIG. 4) prior to this inhalation phase (FIG. 5).

In the above configurations, the opening arrangement and the valve positions may vary since the figures are given and referred to simply as an example. For instance, the interface (4) could be positioned in the upper horizontal part of the storage zone. Also, for instance, the interface can be a Tee-shaped part so that the valve (18) is situated on the opening facing the opening (11) designed to be connected to the patient. The other opening of the Tee-shaped part is connected to the storage zone and arranged on an axis perpendicular to the axis connecting the two other openings. Similarly, the valve (18) can be separated from the interface (4). For instance, a facemask can be connected to the previously defined Tee-shaped part. Furthermore, it is noteworthy that there is no valve on the path of the aerosol as it is administered to the patient, thus eliminating the loss of medicine particles by deposition on the valve during the patient inhalation phase. Nebulization by the mesh is currently obtained by a piezo-electric device, but the use of another technology to put the liquid or the mesh into movement could be applied to this type of mesh nebulizer device. As an example, we refer to the use of a pressure sensor in the container used in the Respimat® dosimetric aerosol produced by Boehringer Ingelheim.

The evident advantages of the invention accurately address the objectives of reducing the loss of aerosols.

The various embodiments described above can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A nebulizer device including a mesh aerosol generator combined with an inhalation chamber and a container designed to contain the medicine in liquid form with mesh connected to the container and in contact with the medicine and allowing the passage of the medicine then the generation of particles of medicine on the other side of the mesh in the inhalation chamber, characterized in that the inhalation chamber is formed with three openings and one single valve, said valve being a one-way expiratory valve, a said first opening being connected to the patient and used for the transport and administration of the particles from the device to the patient, the said first opening being placed upstream of the aerosol generator, a second opening placed upstream of the device aerosol generator and allowing the passage of air from the outside of the chamber to the inside of the inhalation chamber, and allowing the complete ventilation of the inside of the inhalation chamber, a third opening, placed downstream of the aerosol generator and upstream of the said first opening, provided with a single one-way expiratory valve allowing the exhalation of the air from the patient through this third opening, the said third opening being closed by means of the one-way expiratory valve during the inhalation phases and open during the exhalation phases, and whereby the resistance of the said third opening, combined with that of the expiratory valve is less than the resistance of the said second opening in order to limit the escape of the particles produced in the inhalation chamber, through the said second opening, and whereby the second opening is designed to resist the air inhaled by the patient and limit the rate of the air inhaled by the patient.

2. The device according to claim 1, wherein the expiratory valve consists of a shape-memory material which closes it at ambient pressure.

3. The device according to claim 1, wherein the expiratory valve is made from solid non-deformable material.

4. The device according to claim 1 wherein the volume included between the first opening and the third opening is less than 200 mL.

5. The device according to claim 1 wherein the resistance of the third opening and of the expiratory valve is less than 0.07 cm H20 min/L

6. The device according to claim 1, wherein the resistance of the third opening combined with that of the one way expiratory valve is less than the resistance of the second opening for exhalation rate in excess of 3 L/min

7. The device according to claim 1 wherein the resistance of the third opening provided with a one-way expiratory valve is at least 10 times less than the resistance of the second opening for an exhalation rate in excess of 30 L/min.

8. The device according to claim 1 wherein the volume of the inhalation chamber is included between 40 mL and 500 mL

9. A device according to claim 1, wherein the aerosol generator and the first opening are both arranged in the upper part of the device and whereby the storage zone consists of two cylinders and included in one another and arranged according to a vertical axis with the said first cylinder being open at either end and the said second cylinder containing the first cylinder comprises simply one opening in its upper section and whereby the said first cylinder has a diameter smaller than that of the second cylinder and whereby the second cylinder includes the first cylinder and the third opening is situated in the upper part of the device.

10. The device according to claim 9 wherein it has an interface in the upper part of the storage zone near the aerosol generator and includes the said third opening and the expiratory valve which is non-deformable, fitted with a rotation axis hinge opening of the opening during the exhalation phase and closing the opening during the installation phase and during the respiratory pause phases.

11. The device according to claim 10, wherein the interface is a mouthpiece.

12. The device according to claim 9 wherein the first cylinder has a diameter of between 20 mm and 100 mm and a minimum height of 40 mm and whereby the second cylinder has a diameter three times smaller than the diameter of the first cylinder and whereby the second opening has a diameter included between 1 mm and 20 mm.

13. The device according to claim 1 wherein the second opening has a diameter included between 1 mm and 20 mm.

14. The device according to claim 1, wherein the third opening provided with a one-way expiratory valve has a resistance of less than 0.03 cm H20 min/L for an exhaled air rate of 30 L/min.

15. The device according to claim 1, wherein the second opening is located upstream of the first opening, while the third opening is located downstream of the second opening and upstream of the first opening, and whereby the mesh is placed downstream of the second opening and upstream of the third opening.

16. The device according to claim 1 wherein a piezo-electric device is associated with container and not connected to mesh to put into movement the liquid medicine and generate particles through the mesh.

17. The device according to claim 1, wherein a piezo-electric device is connected to the mesh in order to put it into movement and generate particles through the mesh.

18. A device according to claim 16, wherein the vibration frequency of the piezo-electric device is between 50 kHz and 500 kHz.
